(19) United States
(12) Patent Application Publication
(10) Pub. No.: US 2023/0016662 A1
(43) Pub. Date: Jan. 19, 2023

(54) PRE-FILLED SYRINGE STOPPER RETAINER AND METHODS OF USING SAME

(71) Applicants: Ophelia WELLS, Rahway, NJ (US); Wail RASHEED, Rahway, NJ (US); Raymond K. SETO, Rahway, NJ (US); Cassie MEGNA, Rahway, NJ (US); Eduardo CRISTOFOLLI, Rahway, NJ (US); Merck Sharp & Dohme LLC, Rahway, NJ (US)

(72) Inventors: Ophelia Wells, Easton, PA (US); Wail Rasheed, Union, NJ (US); Raymond K. Seto, Warren, NJ (US); Cassie Megna, New Milford, NJ (US); Eduardo Cristofolli, Elizabeth, NJ (US); Michael F. Tormey, Princeton Junction, NJ (US)

(73) Assignee: Merck Sharp & Dohme LLC, Rahway, NJ (US)

(21) Appl. No.: 17/782,955

(22) PCT Filed: Dec. 11, 2020
(86) PCT No.: PCT/US2020/064423

§ 371 Date: Jun. 6, 2022

Related U.S. Application Data
(60) Provisional application No. 62/948,533, filed on Dec. 16, 2019.

(51) Int. Cl. A61M 5/00; B01L 9/00

(52) U.S. Cl. CPC A61M 5/008; B01L 9/54; B01L 2200/025; B01L 2200/18; B01L 2300/041

(57) ABSTRACT

A stopper retainer comprises a body having an upper surface, a lower surface, and at least one fastener, and a plurality of projections extending from the lower surface of the body, each of the plurality of projections being sized to at least partially fit within a lumen of a syringe barrel.

### FIELD OF THE INVENTION

The present disclosure relates generally to storing and transporting syringes. More specifically, the present disclosure relates to a closure device and related methods that allow for safer storage and transportation of pre-filled syringes.

### BACKGROUND OF THE INVENTION

A pre-filled syringe typically includes a glass barrel containing a pharmaceutical product, which is sealed by a stopper. The stopper creates a sterile barrier between the product and the external environment, and is designed to maintain container closure integrity through precise contact with the glass barrel. Gas bubbles within the pre-filled syringe can expand or contract in response to changes in ambient pressure, causing the stopper in the syringe to move from its original position. This movement of the stopper may encourage contamination ingress and the loss of sterility of the product.

Thus, there exists a need for devices that improve upon and advance the methods of safely storing and transporting syringes, such as pre-filled syringes.

### SUMMARY OF THE INVENTION

In one embodiment, a stopper retainer comprises a body having an upper surface and a lower surface, and at least one fastener; and a plurality of projections extending from the lower surface of the body, each of the plurality of projections being sized to at least partially fit within a lumen of a syringe barrel.

In another embodiment of the above stopper retainer, each of the plurality of projections is conical and comprises a proximal end having a largest diameter adjacent the body, and a distal end having a smallest diameter farthest from the body.

In another embodiment of the above stopper retainer, each of the plurality of projections is cylindrical.

In another embodiment of the above stopper retainer, each of the plurality of projections is step-shaped and comprises at least two different diameters.

In another embodiment of the above stopper retainer, the plurality of projections is arranged in rows and columns.

In another embodiment of the above stopper retainer, each column is offset with respect to immediately adjacent columns.

In another embodiment of the above stopper retainer, wherein the stopper retainer comprises four fasteners.

In another embodiment of the above stopper retainer, the four fasteners are arranged with two fasteners being disposed on each of two opposing edges of the body.

In another embodiment of the above stopper retainer, each of the at least one fastener comprises a U-shaped clip configured to be friction fit within a tub.

In another embodiment, the above stopper retainer further comprises a pair of cutouts, each of the pair of cutouts being disposed on a side of the body.

In one embodiment, a system of storing syringes comprises a tub having an interior void; a tray having a plurality of apertures; and the stopper retainer described above; wherein each of the plurality of apertures of the tray is directly aligned with one of the plurality of projections of the stopper retainer.

In another embodiment of the above system, the tray comprises a first number of apertures, and the stopper retainer comprises a second number of projections, the first number and the second number being equal.

### BRIEF DESCRIPTION OF THE DRAWINGS

Various embodiments of the presently disclosed stopper retainers are disclosed herein with reference to the drawings, wherein:

FIGS. 1A-B are schematic illustrations of a pre-filled syringe;

FIGS. 2A-B are schematic illustrations showing a possible movement of a stopper during transportation;

FIG. 3 is a schematic illustration of a tray of syringes being placed in a tub;

FIGS. 4A-C are schematic illustrations of one embodiment of a stopper retainer;

FIG. 4D is a schematic illustration of several variations of projections of a stopper retainer;

FIG. 5 is a schematic illustration of one embodiment of a stopper retainer for use with a tub and a tray; and

FIG. 6 is a schematic cross-section view showing the use of a stopper retainer.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to storage and transporting solutions for pre-filled syringes, conventional methods suffer from some shortcomings as discussed above.

There, therefore, is a need for further improvements to the devices and methods used to help facilitate proper and safer transportation and/or storage of pre-filled syringes. Among other advantages, the present disclosure may address one or more of these needs.

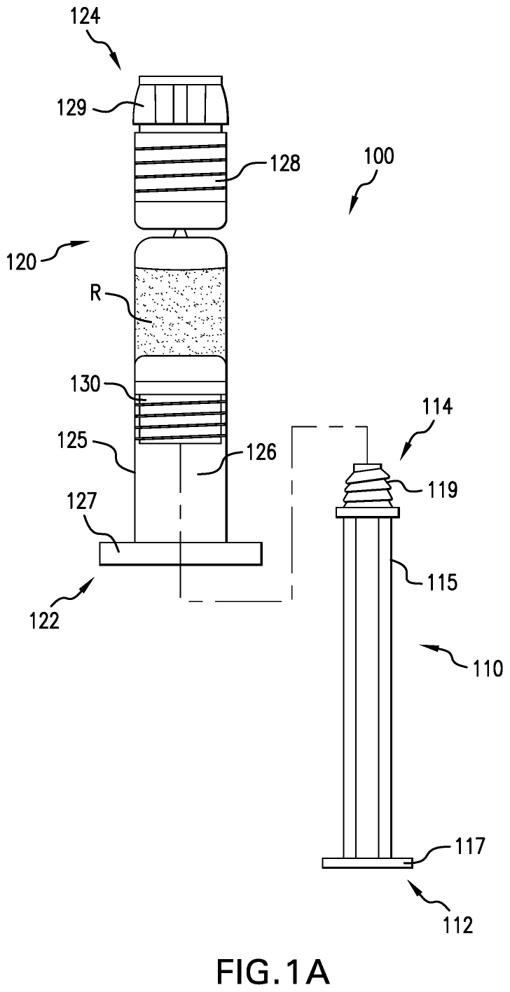

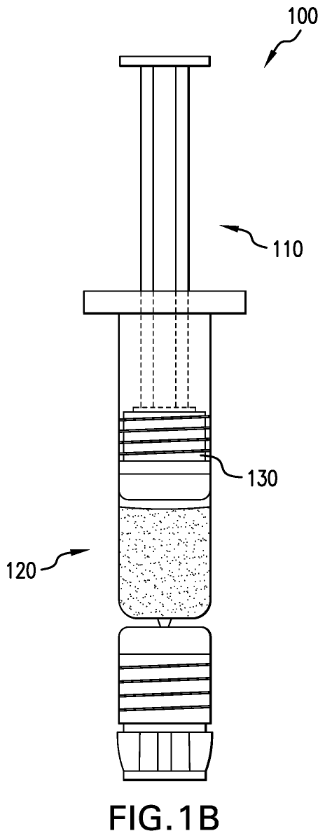

Reference is now made to FIGS. 1A-B, which show schematic illustrations of a prefilled-syringe 100. Pre-filled syringe 100 generally comprises two main portions, a plunger rod 110 and a barrel 120. Plunger rod 110 generally extends between a proximal end 112 and a distal end 114, and comprises an elongated piston 115 extending between a thumb press 117 and a coupler 119. In one embodiment, piston 115 has a cruciform cross-sectional shape.

A cylindrical barrel 120 extends between proximal end 122 and distal end 124 and comprises a body 125 defining a lumen 126 for accepting a portion of plunger rod 110. Body 125 further comprises a flange 127 adjacent proximal end 122, and a removable cap 129 mateable with a hub 128 adjacent distal end 124. Body 125 defines a reservoir “R” that holds a medicament, drug, saline, or other substance. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with coupler 119 of plunger rod 110 by advancing the plunger rod inside the barrel lumen 126 and rotating at least one of coupler 119 and stopper 130 relative to the other. The barrel 120 and the plunger rod 110 may be assembled as shown in FIG. 1B.

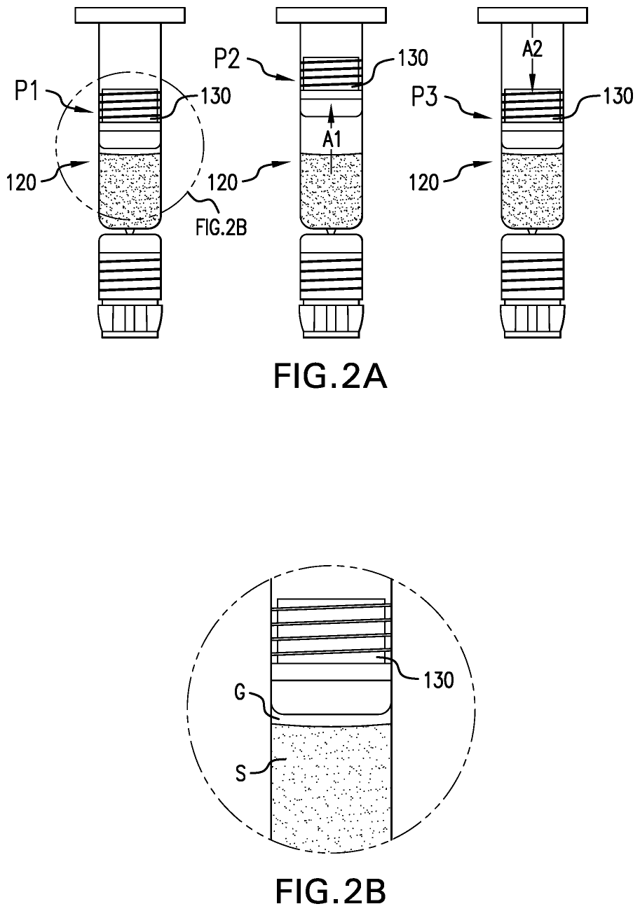

FIGS. 2A-B illustrate how a stopper 130 can move within a barrel 120. When assembled, stopper 130 may be disposed within barrel 120 at position “P1” at ambient temperature and pressure. A gas “G” may be located between the substance “S” and stopper 130. If, during transportation, the barrel is brought to an area of reduced pressure, the gas “G” may expand and stopper 130 may move upward within the barrel in the direction of arrow A1 to position “P2”. This may occur, for example, if the barrel is transported to a location with high elevation and lower atmospheric pressure. If the barrel is subsequently returned to the original pressure, the stopper may move downward within the barrel in the direction of arrow A2 to position “P3”. The movement of the stopper within the barrel may result in contamination ingress and the loss of sterility of the product as previously noted.

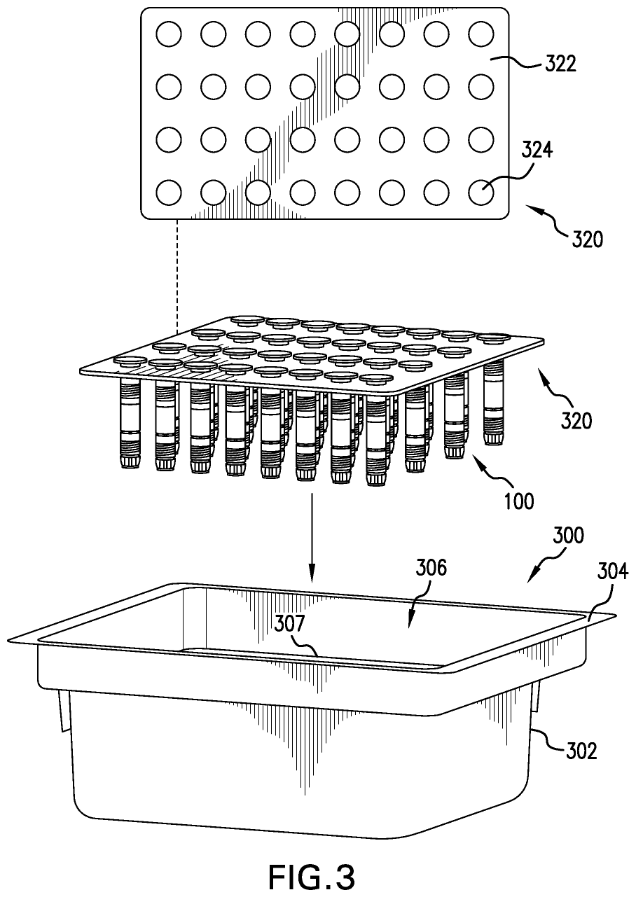

Pre-filled syringes 100 are typically stored and transported in lots disposed in tubs (FIG. 3). Specifically, tub 300 may comprise a body 302 defining a shoulder 304 forming a perimeter and an interior void 306 having a depth capable of accommodating at least a major portion of a syringe barrel. In some embodiments, tub 300 has a generally square or rectangular transverse cross-section. In at least some embodiments, tub 300 has a length of between 5 inches and 36 inches, or more specifically, between 7 inches and 12 inches; a width of between 5 inches and 36 inches, or more specifically, between 6 inches and 10 inches; and a height of between 3 inches and 12 inches, or more specifically, between 3 inches and 5 inches. In some embodiments, tub 300 may accommodate between 1 and 200 syringes.

Tub 300 may be sized to accept a complementary tray 320, the tray having a similar length and/or width as the tub. Tray 320 may be in the form of a flat receptacle that rests on the shoulder of the tub, or may be slightly smaller than the tub and rest at a position within the interior void 306. In at least some embodiments, tub 300 may have a ridge 307 around its interior perimeter and disposed within the interior void 306, and the tray 320 may be sized to rest on this ridge.

In some embodiments, tray 320 may be in the form of a flat receptacle 322 having a number of apertures 324, each aperture having a diameter that is larger than the diameter of the body 125 of the barrel, but smaller than the diameter of the flange 127 of the barrel such that the body 125 of the barrel slides through the aperture, but the flange 127 cannot slide through the apertures 324 and rests securely above the tray. In some embodiments each aperture may have a diameter of between ¼ inch and 2 inches. In the embodiment shown, tray 320 comprises four rows of apertures formed in body 322, each row having eight apertures so that thirty-two barrels can be held by the tray. It will be understood that the shape and/or size of the tub and the tray may be changed as desired to accommodate any number of syringe barrels. Additionally, the number of rows and/or columns, and the number of apertures in each row and/or column may be varied as desired. In some embodiments, tub 300 and tray 320 may both be formed of a plastic, polycarbonate, a metal, or another suitable material.

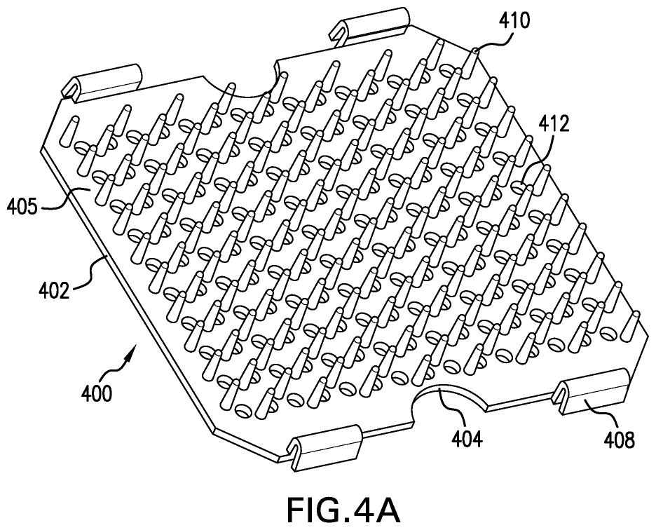

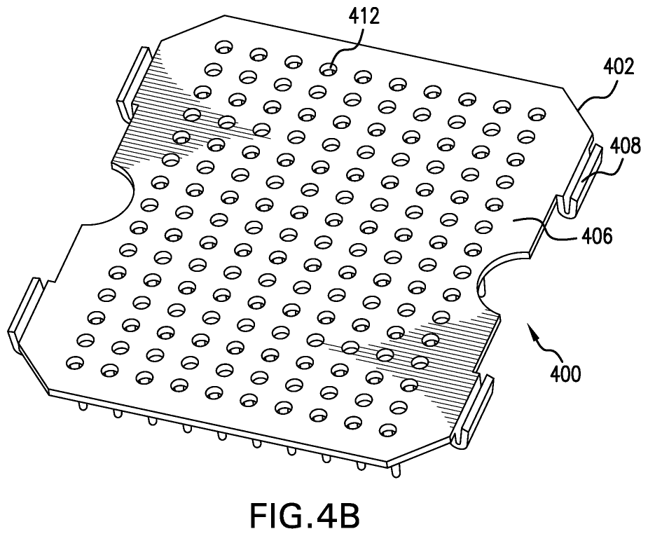

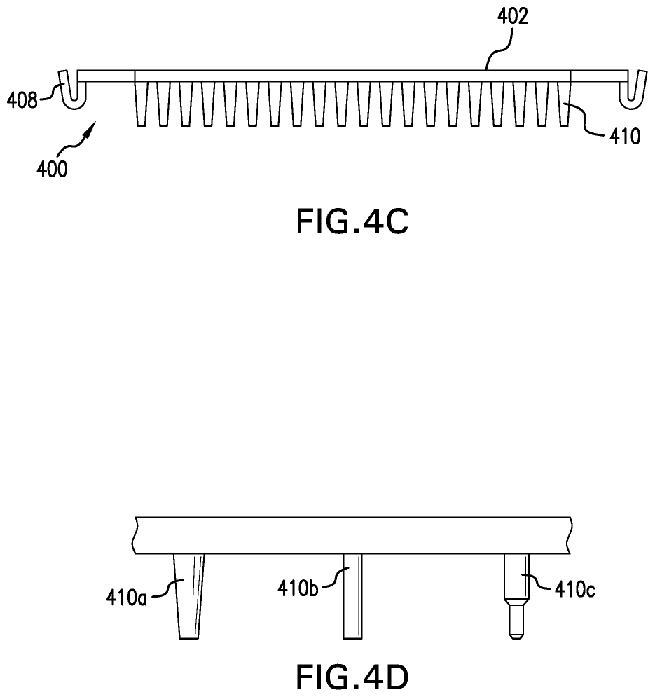

In some embodiments, a stopper retainer may be used to keep the stoppers in place and prevent their movement during transportation and/or storage, the stopper retainer being couplable to a tub and/or tray. In one embodiment, stopper retainer 400 comprises a flat, generally rectangular or square body 402 (FIGS. 4A-C). The shape of the body may be chosen to complement the shape of a tub and/or tray. Body 402 may include a pair of semi-circular, square or triangular cutouts 404 on opposing edges to aid in insertion and removal of the stopper retainer. The cutouts also reduce the weight of the stopper retainer and minimizes the amount of material required for fabrication of the stopper retainer. In some embodiments, more than two cutouts 404 (e.g., pairs of cutouts on each edge) are formed in the body. Body 402 may have a lower surface 405 configured to be disposed closer to the tub and an upper surface 406 configured to be disposed farther from the tub. In the embodiment shown, four clips 408 are used to couple the body to a tub, the clips being disposed on opposing edges of the body with two clips being disposed on each side and a cutout 404 disposed between the two clips 408. In some embodiments, the clips 408 are formed as compressible U-shaped portions that are configured to be friction-fit within a tub. It will be understood that different kinds of clips, fasteners and attachment devices may be used to mate the stopper retainer to a tub and/or tray. Additionally, though the cutouts and clips are disposed on a same edge of the body, other embodiments are possible in which the cutouts and clips are disposed on different edges of the body.

In some embodiments, a number of projections 410 may extend from the lower surface 405 of body 402. Each projection may be approximately 1/32 inch to 2 inches, or more specifically, ¼ inch to 1 inch, in length; and between 1/32 inch and 1 inch, or more specifically, 1/16 inch to 1 inch, in diameter. Each projection may be sized to fit within lumen 126 of the barrel and extend toward the stopper disposed within the barrel to make contact with stopper or terminate immediately adjacent the stopper. The length of the projections may also be dependent on the size of the barrel and/or the quantity of the substance within the barrel.

Various shapes of the projections are possible. FIG. 4D illustrates three variations of the projections. In some embodiments, the projections 410a are cone-shaped similar to those shown in FIGS. 4A-C (i.e., each projection has a decreasing diameter from lower surface 405 toward their distal ends farthest from the lower surface). Instead of being cone-shaped, the stopper retainer may comprise cylindrical projections 410b and have a constant diameter along their height. In a third embodiment, step-shaped projections 410c may include a pair of cylindrical portions with tapered cones forming steps therebetween. It will be understood that all of the projections on a given stopper retainer may be the same, or may include combinations of the shapes described.

The projections 410 may be arranged in rows and columns to reflect the arrangement of the barrels within the tray and the projections may align with the apertures of the tray. In some embodiments, between 2 and 20 rows and between 2 and 20 columns of projections are formed on the lower surface of body 405. In some embodiments, each of the rows of projections may be offset from adjacent rows. In some embodiments, each of the columns of projections may be offset from adjacent columns. Offsetting of rows and/or columns may allow for an increase in the density of the projections and the number of barrels to be transported in a given tub. In some embodiments, a number of holes 412 may be drilled or otherwise formed in interstitial spaces of the body between projections to reduce the weight of the stopper retainer and minimize the material required for fabrication of the stopper retainer. Holes 412 may be circular or may have any other suitable shape.

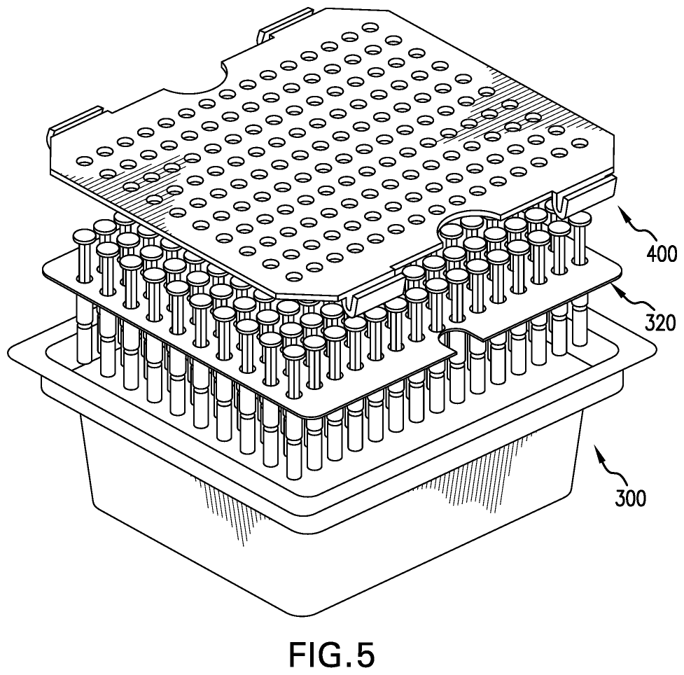

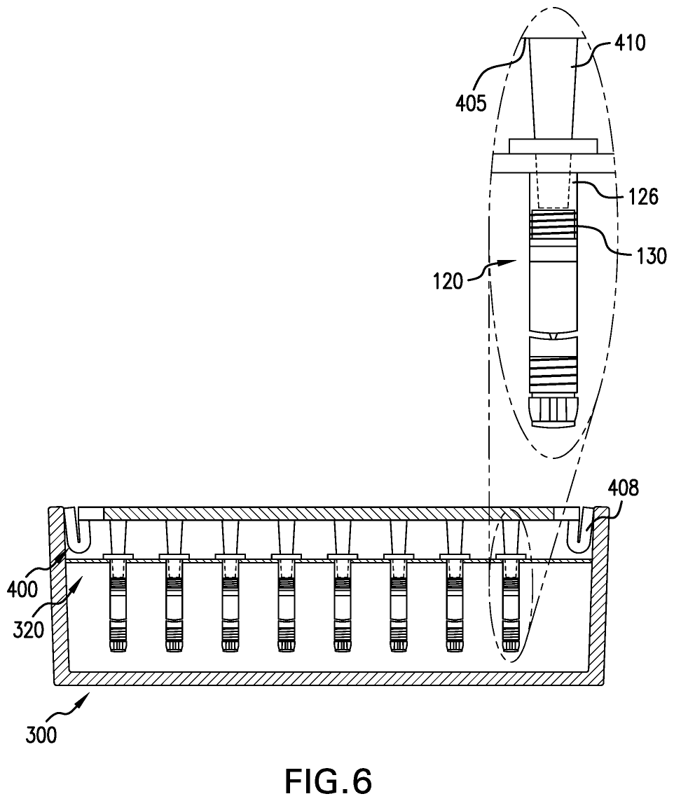

In use, stopper retainer 400 may be provided as part of a kit or system with tub 300 and tray 320 (FIG. 5). In some embodiments, the apertures 324 of tray 320 will easily align with projections 410 of stopper retainer 400, and the number of apertures 324 of tray 320 is the same as the number of projections 410 of stopper retainer 400. With the tray 320 being disposed in the tub 300, the barrels of each syringe may be suspended in, or rest on a surface of, the tub. A cross-sectional view is shown by way of explanation. In the embodiment of FIG. 6, only a first row of syringes is shown as they are secured by a first row of projections 410 of the stopper retainer 400. Each projection 410 extends from lower surface 405 of the body 402, and enters the lumen 126 of the barrel 120, terminating adjacent stopper 130 within the lumen 126 of the barrel 120. Projection 410 may contact the stopper 130 or may terminate immediately adjacent the stopper (e.g., within ⅛ of an inch from the stopper or some other specific tolerance). With projections 410 being disposed in each of the barrels 120, and with clips 408 friction fit within tub 300, the projections will prevent the stoppers from moving upward upon the expansion of gas when the ambient pressure is reduced relative to the internal pressure of the syringe, reducing the risk of contamination and/or loss of sterility. Moreover, the clips 408 will keep the stopper retainer in place and immovable with respect to the tub.

It is to be understood that the embodiments described herein are merely illustrative of the principles and applications of the present disclosure. For example, the number and arrangement of projections may be varied. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A stopper retainer, comprising:
a body having an upper surface and a lower surface, and at least one fastener; and
a plurality of projections extending from the lower surface of the body, each of the plurality of projections being sized to at least partially fit within a lumen of a syringe barrel.

2. The stopper retainer of claim 1, wherein each of the plurality of projections is conical and comprises a proximal end having a largest diameter adjacent the body, and a distal end having a smallest diameter farthest from the body.

3. The stopper retainer of claim 1, wherein each of the plurality of projections is cylindrical, triangular, spherical or rectangular.

4. The stopper retainer of claim 1, wherein each of the plurality of projections is step-shaped and comprises at least two different diameters.

5. The stopper retainer of claim 1, wherein the plurality of projections is arranged in rows and columns.

6. The stopper retainer of claim 5, wherein each column is offset with respect to immediately adjacent columns.

7. The stopper retainer of claim 1, wherein the stopper retainer comprises four fasteners.

8. The stopper retainer of claim 7, wherein the four fasteners are arranged with two fasteners being disposed on each of two opposing edges of the body.

9. The stopper retainer of claim 1, wherein each of the at least one fastener comprises a U-shaped clip configured to be friction fit within a tub.

10. The stopper retainer of claim 1, further comprising a pair of cutouts, each of the pair of cutouts being disposed on a side of the body.

11. A system of storing syringes comprising:
a tub having an interior void;
a tray having a plurality of apertures; and
the stopper retainer of claim 1;
wherein each of the plurality of apertures of the tray is directly aligned with one of the plurality of projections of the stopper retainer.

12. The system of claim 11, wherein the tray comprises a first number of apertures, and wherein the stopper retainer comprises a second number of projections, the first number and the second number being equal.
